(19) United States
(12) Patent Application Publication
(10) Pub. No.: US 2011/0008863 A1
(43) Pub. Date: Jan. 13, 2011

(54) Methods for Producing Fermentation Products

(75) Inventors: Yongming Zhu, Wake Forest, NC (US); Mads Peter Torry Smith, Raleigh, NC (US); Brandon Cory Emme, Youngsville, NC (US)

Correspondence Address: NOVOZYMES NORTH AMERICA, INC., 500 FIFTH AVENUE, SUITE 1600, NEW YORK, NY, 10110, US

(73) Assignee: NOVOZYMES NORTH AMERICA, INC., Franklinton, NC (US)

(21) Appl. No.: 12/920,655

(22) PCT Filed: May 18, 2009
(86) PCT No.: PCT/US2009/044333

§ 371 Date: Sep. 2, 2010

Related U.S. Application Data
(60) Provisional application No. 61/053,724, filed on May 16, 2008.

(51) Int. Cl. C12P 7/10; C12P 1/02; C12P 1/00; C08L 97/02

(52) U.S. Cl. 435/165; 435/171; 435/41; 530/500

(57) ABSTRACT

The invention relates to methods for treating pre-treated lignocellulose-containing material comprising the steps of: a) subjecting a slurry comprising pre-treated lignocellulose-containing material to agitation in the presence of one or more chemicals and/or one or more enzymes; b) subjecting said slurry to liquid-solid separation; c) recycling at least a portion of the liquid to the agitated slurry; d) optionally transferring solids-containing material for downstream processing.

### TECHNICAL FIELD

The present invention relates to methods for treating pre-treated lignocellulose-containing material in order to ease handling of the material and/or downstream processing, e.g., hydrolysis of high solids lignocellulose-containing material slurries. The invention also related to processes of producing a fermentation product from lignocellulose-containing material including a treatment method of the invention.

### BACKGROUND OF THE INVENTION

Inexpensive lignocellulose-containing feed stock is available in abundance and can be used for producing renewable fuels such as ethanol. Producing fermentation products from lignocellulose is known in the art and generally includes pre-treating, hydrolyzing and fermenting the material.

The structure of lignocellulose is not directly accessible to enzymatic hydrolysis. Therefore, the lignocellulose-containing material is pre-treated in order to break the lignin seal and disrupt the crystalline structure of cellulose. This may cause solubilization and saccharification of the hemicellulose fraction. The cellulose fraction is then hydrolyzed enzymatically, e.g., by cellullolytic enzymes, which degrades the carbohydrate polymers into fermentable sugars. These fermentable sugars are then converted into the desired fermentation product by a fermenting organism.

High solids pre-treated lignocellulose-containing material (i.e., pre-treated biomass) has a high viscosity. This makes handling of the lignocellulose-containing material during downstream processing, such as enzymatic hydrolysis, extremely difficult and thus inefficient and costly.

Consequently, there is a need to solve the handling problem for high solids pre-treated lignocellulose-containing material slurries.

### SUMMARY OF THE INVENTION

Handling slurries containing a high solids content of pre-treated lignocellulose-containing material represents a major problem and can be expensive due to high energy costs and/or inefficient. The present invention relates to methods for managing and solving this problem.

In the first aspect the invention relates to methods for treating pre-treated lignocellulose-containing material comprising the steps of:

a) subjecting a high solids slurry comprising pre-treated lignocellulose-containing material to dilution and agitation in the presence of one or more chemicals and/or one or more enzymes;

b) subjecting the treated slurry from step a) to liquid-solid separation;

c) recycling at least a portion of the liquid separated from step b) for diluting a slurry as described in step (a);

d) optionally transferring the solids-containing material coming from step b) for downstream processing.

The invention may result in a number of advantages including, but not limited to: more even enzyme distribution across the available surface area of substrate; more effective pH and/or temperature control that may result in higher fermentation yields; reduced viscosity of pre-treated lignocellulose substrate is promoted making transportation, storage and handling of lignocellulose-containing materials easier and improves mixing in of the chemicals(s) and/or enzyme(s), reduces minimum water content in the hydrolysis step, and reduces distillation costs in a lignocellulose-to-ethanol process; allows use of industrially established agitation/mixing apparatus and liquid/solid separation equipment; allows continuous operation; if desired, the solid content after the solid-liquid separation can be easily controlled; and saves energy.

In a second aspect the invention relates to processes of producing fermentation products from lignocellulose-containing material comprising the steps of:

i) pre-treating lignocellulose-containing material;

ii) treating the pre-treated lignocellulose-containing material according to a method of the invention;

iii) hydrolyzing the material obtained in step ii); and

iv) fermenting using one or more fermenting organisms.

### BRIEF DESCRIPTION OF THE FIGURES

FIG. 1 shows a one-vessel agitated system in which the agitated vessel is used for adding chemical and/or enzyme.

FIG. 2 shows a two-vessel agitated system in which the first agitated vessel is used for addition of chemicals and a second agitated vessel following the first agitated vessel but before solid-liquid separation is used for adding enzyme.

FIG. 3 shows a two-vessel agitation system in which a first agitated vessel is used for adding chemicals and a second agitated vessel following solid-liquid separation is used for adding enzyme.

FIG. 4 shows a modification of the system in FIG. 1 where an immobilized mediator has been introduced between solid-liquid separation and the agitated slurry.

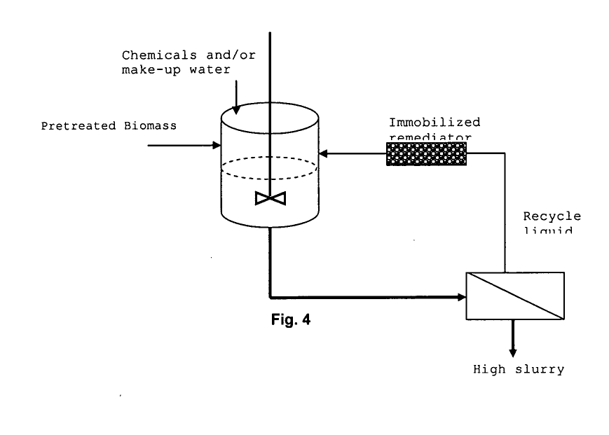

FIG. 5 shows the sugar concentration in simulation reactors and control after 24 hours.

FIG. 6 shows the sugar concentration in simulation reactors and control after 72 hours.

### DETAILED DESCRIPTION OF THE INVENTION

The invention relates to methods of treating pre-treated lignocellulose-containing material in order to ease handling of high solids pre-treated lignocellulse-containing material slurries. This is at least partly done by obtaining improved distribution of chemicals and/or enzymes, especially hydrolytic enzymes, used in downstream processing. The method of the invention can alternatively or additionally be used for improving distribution of chemicals for, e.g., effectively controlling pH and/or other process conditions. High solids slurries generally mean slurries with a content of insoluble solids above 10 wt. %, typically from 10-80 wt. %, such as 10-50 wt. % preferably around 25 wt. %.

The term “improving distribution” means that contact between the chemical and/or enzyme(s) and the lignocellulose-containing material is improved. The improved distribution can lead to, e.g., faster and/or more effective (less enzyme needed) enzymatic hydrolysis during downstream processing compared to processes where a method of the invention has not been employed.

In the first aspect the invention relates to methods for treating pre-treated lignocellulose-containing material comprising the steps of:

a) subjecting a high solids slurry comprising pre-treated lignocellulose-containing material to dilution and agitation in the presence of one or more chemicals and/or one or more enzymes;

b) subjecting the treated slurry from step a) to liquid-solid separation;

c) recycling at least a portion of the liquid separated from step b) for diluting a slurry as described in step (a);

d) optionally transferring the solids-containing material coming from step b) for downstream processing.

The solids-containing material coming from solid-liquid separation in step b) may be subjected to further treatment before being transferred for downstream processing. In an embodiment the further treatment includes the steps of:

e) subjecting all or part of the solids-containing material obtained from the liquid-solid separation in step b) or f) to dilution and agitation in the presence of one or more chemicals and/or one or more enzymes;

f) subjecting the material from step e) to liquid-solid separation;

g) recycling at least a portion of the liquid separated from step f) for diluting a slurry as described in step (a) or the solids-containing material described in step e);

h) transferring the solids-containing material coming from step f) for downstream processing.

It is also contemplated that steps a) through g) may be repeated one or more times before the solids-containing material is transferred for downstream processing.

It is to be understood that the solids-containing material obtained from step b) or step f) comprises lignocellulose-containing material and can be referred to as such in accordance with the invention.

In one embodiment of the invention the recycled liquid coming from the solid-liquid separation in step b) or step f) for recycling as described in step c) or step g) is subjected to conditioning, such as detoxification, prior to being reintroduced into the method of the invention. This may be done in any suitable way well know in the art. In one preferred embodiment an immobilized remediator is inserted after step b) and/or step f) and before step c) and/or step g). The term “conditioning” has, according to the invention, its art-recognized meaning which includes treatments that enhance process performance downstream, e.g., hydrolysis and/or fermentation, or other downstream processes such as sulphur and/or carboxylic acids removal done to prolong process equipment lifetime or ease of processing. Additional conditioning may comprise pH or temperature adjustment of the liquid, or the removal, modification, or recapture of compounds contained in the recycled liquid. In an embodiment, the recycled liquid can be used for diluting the lignocellulose-containing material in step a) and/or step e).

In preferred embodiments, agitation is carried out in a mixing tank, vessel, pump or the like. However, another kind of equipment may also be use. In a preferred embodiment the liquid comprises any suitable liquid such as water or buffers.

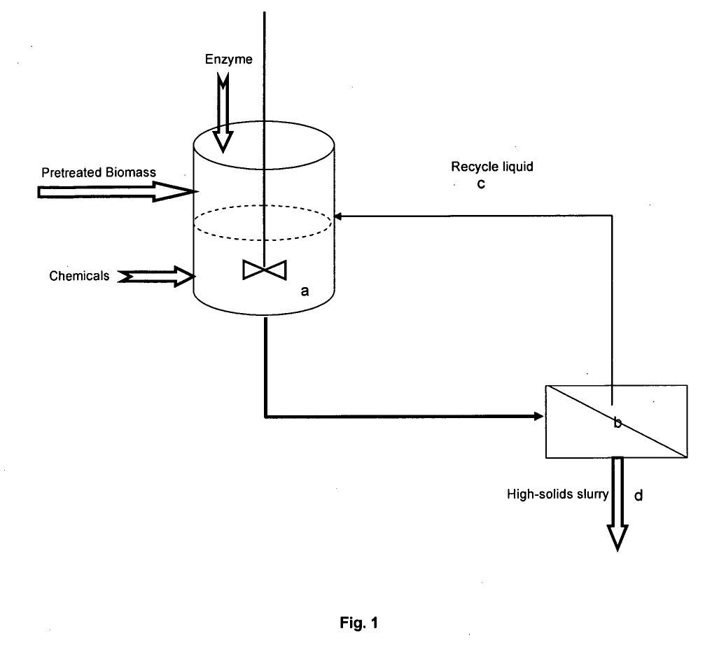

Generally, lignocellulose-containing material is added to suitable equipment in order to prepare an aqueous slurry comprising mainly pre-treated lignocellulose-containing material and water or buffer. One or more chemicals and/or one or more enzymes are added to the agitated slurry in step a) before solid-liquid separation in step b). In step c), at least a portion of the liquid from the separation in step b) is recycled to the agitated slurry of step a), and the solids-containing material from the liquid-solid separation of step b) is optionally transferred for downstream processing (step d)) (see, e.g., FIG. 1).

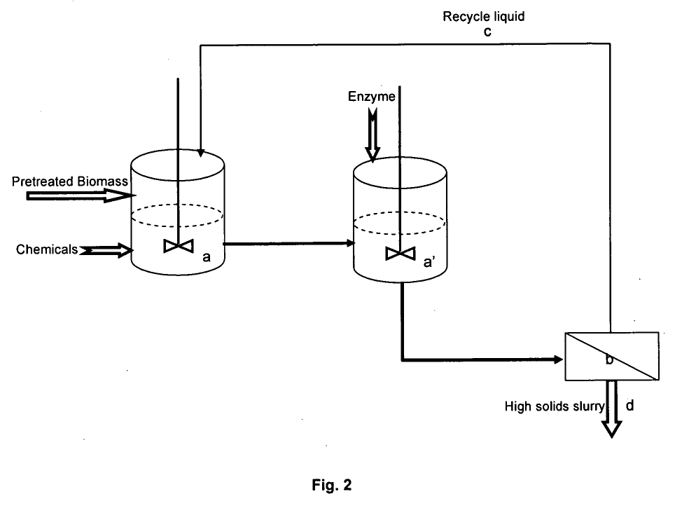

In an embodiment one or more chemicals are added during step a) and at least one or more enzymes are added in step a) or in a separate step a′) after step a), but before solid-liquid separation in step b). After solid-liquid separation in step b) at least a portion of the liquid is recycled in step c) to the agitated slurry (in step a) and/or step a′)), and the solids-containing material coming from step b) is optionally transferred for downstream processing (see, e.g., FIG. 2).

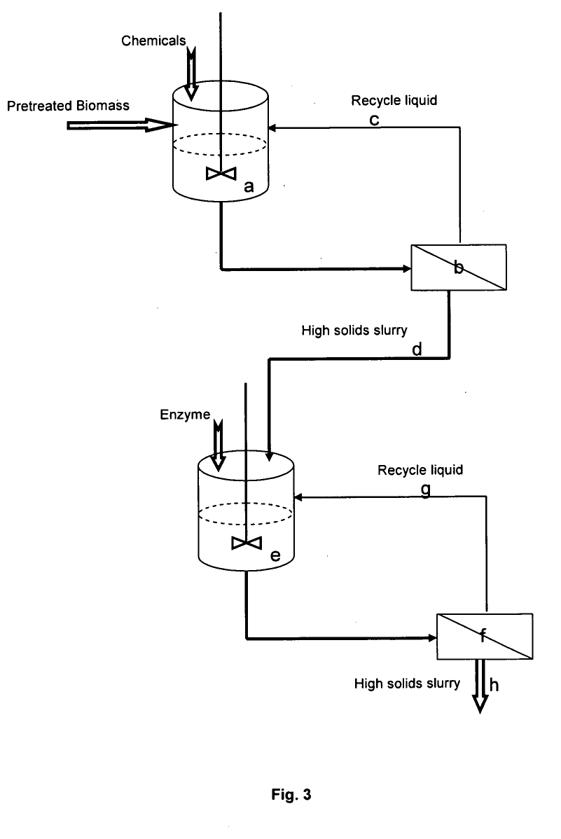

In a further embodiment one or more chemicals and/or one or more enzymes are introduced into the agitated slurry in step a) followed by solid-liquid separation in step b), recycling of at least a portion of the liquid in step c) to the agitated slurry (in step a)). The solids-containing material coming from solid-liquid separation is then subjected to dilution and agitation in the presence of one or more chemicals and/or one or more enzymes in step e), before solid-liquid separation in step f). The solids-containing material is transferred for downstream processing in step h) (see, e.g., FIG. 3).

The lignocellulose-containing material content in step a) and/or step a′) and/or e) following dilution are generally kept low and may constitute from between 0.5-15 wt. %, preferably 1-10 wt. % insoluble solids of the slurry. It should be understood that one or more additional agitation steps may take place, i.e., more than one or two agitation steps, i.e., in steps a) or a′) or e). During additional agitation step(s) chemicals and/or enzymes may be introduced.

In a preferred embodiment the enzyme(s) added during step a) and/or step a′) and/or e) is(are) hydrolytic enzyme(s). The enzyme(s) added to the aqueous lignocellulose containing slurry are preferably hydrolytic enzyme(s) selected from the group consisting of cellulolytic enzymes, hemicellulolytic enzymes, amylolytic enzymes, pectolytic enzymes, proteolytic enzymes, esterase enzymes, or a mixture of two of more thereof. Other polypeptides such as cellulolytic enhancing activity polypeptides, e.g., GH61A polypeptides, may also be present or added together with the hydrolytic enzymes.

In a preferred embodiment downstream processing of the solids-containing material after step d) or h) is or includes an enzymatic hydrolysis step. The chemical(s) may be any suitable chemicals including pH adjusting agents, such as acids, bases, buffers; conditioning agents, such as resins and charcoal; detoxification agents; wetting agent, including surfactants, such as nonionic surfactants.

In one embodiment the method of the invention is initiated in an agitating vessel in which chemical(s) are added for, e.g., pH adjustment and/or conditioning of the slurry, and/or enzyme distribution (FIG. 1). A variation of this is a two-vessel agitated system in which a first vessel is used for addition of chemicals, e.g., pH adjustment and/or conditioning agents, and a second vessel where enzymes are added, e.g., to obtain better enzyme distribution (FIG. 2 or FIG. 3). In both embodiments the pre-treated lignocellulose-containing material slurry from the vessel(s) is(are) subjected to solid-liquid separation; recycling of a liquid fractions to vessel(s) capable of agitation and finally transfer of a high solids-containing material fraction for downstream processing, for instance for hydrolysis and/or fermentation.

The content/concentration of lignocellulose-containing material in the slurry in step a) and/or step a′) and or step e) may be adjusted by the amount of recycled liquid from step b) and/or step f).

In an embodiment the lignocellulose-containing material content leaving step b) and/or f) comprises from between 10-80 wt. % insoluble solids, preferably from between 10-50 wt. %, such as from between 20-40 wt. %, or around 25 wt. % insoluble solids of the total mass flow (i.e., solids and liquids). In other words, the solid fractions in step d) or g) is(are) high solids slurry/slurries.

### Recycling of Liquid and Recycling Ratio

In an embodiment from between 40-99 wt. % liquid, preferably from between 60-80 wt. % liquid of the total mass flow (i.e., solids and liquids) separated in step b) or f) is recycled. The portion of liquid recycled is used to dilute or adjust the solids content in the agitated slurry/slurries.

Generally, the recycling ratio (RR) may be defined as the ratio of the mass flow of the recycle liquid stream (kg/hr) to the mass flow of the total input stream (kg/hr), i.e., which may include fresh pre-treated lignocellulose-containing material substrate stream(s), chemical feed stream(s) and/or enzyme feed stream(s). The calculation equation is:

RR=Si/Sd−1

where Si is the content/concentration of insoluble solids (wt %) in the total input stream(s), and Sd is the content/concentration of insoluble solids (wt %) leaving the agitation vessel (diluted slurry).

Suitable dosing of chemicals and/or enzymes can easily be determined by one skilled in the art. Generally, dosing of enzymes will depend on the dosing desired during downstream processing such as enzymatic hydrolysis and/or fermentation.

In a preferred embodiment at least 0.1 mg cellulolytic enzyme protein per gram insoluble solids, preferably 3 mg cellulolytic enzyme protein per gram insoluble solids is added to the pre-treated lignocellulose-containing material slurry in step a) and/or step a′) and/or step e). If desired a holding stage, e.g., minutes to hours, between agitation and solid-liquid separations may be introduced. The main purpose of the method of the invention is not to actually carry out downstream processing, e.g., hydrolysis, rather to improve downstream processing such as hydrolysis. The purpose may in one embodiment be to improve chemical and/or enzyme contact with the lignocellulose-containing material in order to, e.g., obtain faster hydrolysis and/or higher fermentation yields, or reduce chemicals and/or enzymes necessary in a process of the invention.

In an embodiment cellulolytic enzyme(s) are dosed in the range from 0.1-100 FPU per gram insoluble solids, preferably 0.5-50 FPU per gram insoluble solids, especially 1-30 FPU per gram insoluble solids.

In an embodiment hemicellulolytic enzyme(s) is(are) present/added to the agitated slurry in step a) and/or step a′) and/or step e). Treatment in step a) and/or step a′) and/or step e) may preferably be carried out at a pH in the range from 4-8, preferably 5-7. pH adjusting agents may be used to accomplish that. Agitation in step a) and/or step a′) and/or step e) may preferably be carried out at a temperature in the range from 20-70° C., preferably from 25-60° C.

The solids-containing material coming from step b) and/or f) may be transferred for downstream processing. Downstream processing includes simultaneous hydrolysis and fermentation (SSF); hybrid hydrolysis and fermentation (HHF); or separate hydrolysis and fermentation (SHF).

The period that the lignocellulose-containing material is subjected to agitation (retention time) can easily be determined by one skilled in the art and depends at least to some extent on the equipment used. The retention time in step a) and/or step a′) and/or step e) may typically be from 1 second to 1 hour, preferably 2 seconds to 10 minutes. In the case of high shear mixing the retention time generally is from between 1 second to 10 minutes.

The diluted pre-treated lignocellulose-containing material content in step a) and/or step a′) and/or e) may comprise from between 0.5-15 wt. % of the slurry determined as insoluble solids, preferably 1-10 wt. %.

### Agitation

The term “agitation” has its art-recognized meaning in context of the present invention and includes any kind of mixing of the pre-treated lignocellulose-containing material slurries and chemical(s) and/or enzyme(s). For instance, agitation also includes transportation/transferring of material using a pump.

According to the invention agitation includes all from low speed mixing to high speed or high shear mixing. Generally, agitation can be defined in, e.g., Reynolds numbers as will be explained further below.

Due to a low (0.5-15 wt. %) insoluble solids content prior to dilution in the agitated slurry in, e.g., step a) the pretreated lignocellulose-containing material may be subjected to homogenization, wet milling or the like. In an embodiment the pretreated lignocellulose-containing material may be subjected to high-shear disintegration of lignocellulose, for instance, using high-frequency or a rotor stator device to microcavitate the slurry to shatter the fibrous structure of the lignocellulose.

Generally, the goal of agitating the lignocellulse-containing material is to improve wetting and/or contact between chemical(s) and/or enzyme(s) and the pre-treated lignocellulose-containing material. Agitation can also be used, for instance, for pH adjustment.

Agitation equipment is generally well-known in the art.

Mixers that can agitate at low speed or high speed are well known in the art. High speed mixers are often referred to as high shear mixers, high speed mixers or high shear granulators, or the like. Examples of such mixers/granulators are disclosed in, e.g., Remington: The Science and Practice of Pharmacy, 19th Edition (1995) or in Handbook of pharmaceutical granulation technology, chapter 7, “Drugs and the pharmaceutical sciences”, vol. 81, 1997. High shear mixers may be selected from the following types: Gral, Lodige/Littleford, Diosna, Fielder or Baker-Perkins.

### Reynolds Number

In fluid mechanics, the Reynolds number is the ratio of inertial forces (vₛρ) to viscous forces (μ/L) and consequently quantifies the relative importance of these two types of forces for given flow conditions. Thus, it is used to identify different flow regimes, such as laminar or turbulent flow. It is one of the most important dimensionless numbers in fluid dynamics and is used, usually along with other dimensionless numbers, to provide a criterion for determining dynamic similitude. When two geometrically similar flow patterns, in perhaps different fluids with possibly different flowrates, have the same values for the relevant dimensionless numbers, they are said to be dynamically similar.

Typically Reynolds number is given as follows:

$$Re = \frac{ρ {v}_{s}^{2} / L}{μ {v}_{s} / {L}^{2}} = \frac{ρ {v}_{s} L}{μ} = \frac{{v}_{s} L}{v} = \frac{Inertial forces}{Viscous forces}$$

where:
vₛ—mean fluid velocity, [m s⁻¹]
L—characteristic length, [m]
μ—(absolute) dynamic fluid viscosity, [N s m⁻²] or [Pa s]
v—kinematic fluid viscosity: v=μ/ρ, [m² s⁻¹]
ρ—fluid density, [kg m⁻³].

For flow in a pipe for instance, the characteristic length is the pipe diameter, if the cross section is circular, or the hydraulic diameter, for a non-circular cross section.

Laminar flow occurs at low Reynolds numbers, where viscous forces are dominant, and is characterized by smooth, constant fluid motion, while turbulent flow, on the other hand, occurs at high Reynolds numbers and is dominated by inertial forces, producing random eddies, vortices and other flow fluctuations.

According to one embodiment of the invention agitation in step a) and/or step a′) may be carried out to provide a laminar agitation flow. Laminar agitation may according to the invention be done at 1,500 Re or below 1,500 Re (Reynolds number).

In a preferred embodiment agitation in step a) and/or step a′) is carried out to provide a turbulent agitation flow. Turbulent agitation may according to the invention be done at above 1,500 Re (Reynolds number), preferably above 2,100 Re, especially. In a preferred embodiment agitation is carried out as high shear mixing at Re from between 2,100-30,000 Re, such as between about 5,000-20,000 Re. In an embodiment the high shear mixing results in fluidization of the lignocellulose-containing material.

FIG. 1 shows a suitable equipment setup/system for carrying out methods of the invention where pre-treated lignocellulose-containing material is continuously or batchwise fed to an agitation vessel. The lignocellulose-containing material content of insoluble solids is generally kept low (e.g., 0.5-15 wt. % of the slurry). Chemical(s) and/or enzyme(s) may be added to the agitation vessel. A lignocellulose-containing material stream is transported/transferred from said vessel to solid-liquid separation equipment. At least a fraction of the liquid stream coming from the solid-liquid separation equipment is recycled to the agitating vessel(s). A high-solids stream (10-80 wt. % insoluble solid material) coming from the solid-liquid separation equipment may optionally be transported/transferred for downstream processing, for instance, to vessels suitable for hydrolysis and/or fermentation of pre-treated lignocellulose-containing material.

FIG. 2 shows another suitable equipment setup/system for carrying out methods of the invention wherein pre-treated lignocellulose-containing material is continuously or batchwise fed to an agitation vessel. The lignocellulose-containing material content of insoluble solids is kept low (e.g., 0.5-15 wt. % of the slurry). Chemical(s) is(are) added to a first agitation vessel. The lignocellulose-containing material stream is transported/transferred to a second agitation vessel where enzyme(s) may be added. A stream from said second vessel is transported/transferred to solid-liquid separation equipment. At least a portion/fraction of the liquid stream coming from the solid-liquid separation equipment is recycled to one or both of the two agitation vessels. A high-solids stream (10-80 wt. % insoluble material) coming from the solid-liquid separation equipment is optionally transferred for downstream processing, for instance, to a vessel suitable for hydrolysis and/or fermentation of lignocellulose-containing material.

FIG. 3 shows a third suitable equipment setup/system for carrying out methods of the invention wherein pre-treated lignocellulose-containing material is continuously or batchwise fed to an agitation vessel. The lignocellulose-containing material content of insoluble solids is kept low (e.g., 0.5-15 wt. % of the slurry). Chemical(s) is(are) added to a first agitation vessel. A stream from said first vessel is transported/transferred to solid-liquid separation equipment. At least a portion/fraction of the liquid stream coming from the solid-liquid separation equipment is recycled to the agitated vessel. A high-solids stream (10-80 wt. % insoluble material) coming from the solid-liquid separation equipment is transported/transferred to a second agitation vessel where enzyme(s) may be added. At least a portion/fraction of the liquid stream coming from the second solid-liquid separation equipment is recycled to the agitated vessel. A high-solids stream (10-80 wt. % insoluble material) coming from the second solid-liquid separation equipment is optionally transferred to for downstream processing, e.g., hydrolysis and/or fermentation.

Given the high mixability in the first vessels, due to a low lignocellulose-containing material content (0.5-15 wt. % insoluble solids), the feed flows, chemical and/or enzyme dosing can be done continuously or stepwise.

### Solid-Liquid Separation

Solid-liquid separation can be achieved in many ways well-known to one skilled in the art. For instance, solid-liquid separation can be done using a screw press, centrifugation, belt press, drum filter, hydrocyclone and/or filter press, or any kind of apparatus which can handle solids-liquid separation, including gravity-fed systems or apparatuses.

The separated liquid can then be recycled to the agitated vessel(s), tank(s) or the like, or, in some cases, can be partially withdrawn as a side stream. The latter may be used to control the solid content transferred, e.g., for downstream processing.

### Process of Producing Fermentation Products

In a second aspect the invention relates to processes of producing fermentation products from lignocellulose-containing material comprising the steps of:

i) pre-treating lignocellulose-containing material;

ii) subjecting the pre-treated material using a method of treating pre-treated lignocellulose-containing material of the invention;

iii) hydrolyzing the material coming from step ii); and

iv) fermenting using one or more fermenting organisms.

According to the invention step iii) and fermentation step iv) may be carried out sequentially or simultaneously. The pre-treated lignocellulose-containing material may be wholly or partly hydrolyzed before fermentation is initiated or finalized, respectively. The hydrolysis and fermentation steps may be carried out as simultaneous hydrolysis and fermentation (SSF). Alternatively, steps iii) and iv) may be carried out as hybrid hydrolysis and fermentation (HHF) or separate hydrolysis and fermentation (SHF).

Simultaneous hydrolysis and fermentation (SSF) generally means that hydrolysis and fermentation are combined and carried out at conditions (e.g., temperature and/or pH) suitable for the fermenting organism in question.

Hybrid hydrolysis and fermentation (HHF) generally begins with a separate hydrolysis step, where the lignocellulose is partly (e.g., 10-50%, such as 30% hydrolyzed) and ends with a simultaneous hydrolysis and fermentation step (SSF). The separate hydrolysis step is an enzymatic cellulose saccharification step typically carried out at conditions (e.g., at higher temperature) suitable, preferably optimal, for the hydrolyzing enzyme(s) in question. The subsequent simultaneous hydrolysis and fermentation step (SSF) is typically carried out at conditions suitable for the fermenting organism(s) (often at lower temperature than the separate hydrolysis step). As mentioned above, hydrolysis step iii) may also be carried out separately from the fermentation step iv). In such case lignocellulose is wholly hydrolyzed before fermentation is initiated.

Examples of suitable enzymes and/or hydrolyzing enzymes can be found in the “Enzymes” section below. Suitable process conditions can easily be determined by the skilled artisan.

### Pre-Treatment

The lignocellulose-containing material may be pre-treated in any suitable way.

Pre-treatment is carried out before hydrolysis and/or fermentation. In a preferred embodiment the pre-treated material is hydrolyzed, preferably enzymatically, before fermentation. The goal of pre-treatment is to reduce the particle size, separate and/or release cellulose; hemicellulose and/or lignin and in this way increase the rate of hydrolysis. Pre-treatment methods such as wet-oxidation and alkaline pre-treatment targets lignin, while dilute acid and auto-hydrolysis targets hemicellulose. Steam explosion is an example of a pre-treatment that targets cellulose.

According to the invention the pre-treatment step may be a conventional pre-treatment step using techniques well known in the art. In a preferred embodiment pre-treatment takes place in a slurry of lignocellulose-containing material and water. The lignocellulose-containing material may during pre-treatment be present in an amount between 10-80 wt. % TS, preferably between 20-70 wt. % TS, especially between 30-60 wt. % TS, such as around 50 wt. % TS.

Chemical, Mechanical and/or Biological Pre-Treatment

The lignocellulose-containing material may according to the invention be chemically, mechanically and/or biologically pre-treated before hydrolysis in accordance with the method of the invention. Mechanical pre-treatment (often referred to as “physical”-pre-treatment) may be carried out alone or may be combined with other pre-treatment methods.

Preferably, the chemical, mechanical and/or biological pre-treatment is carried out prior to carrying out a method of the invention.

### Chemical Pre-Treatment

The term “chemical pre-treatment” refers to any chemical pre-treatment which promotes the separation and/or release of cellulose, hemicellulose and/or lignin. Examples of suitable chemical pre-treatments include treatment with for example dilute acid, lime, alkaline, organic solvent, ammonia, sulfur dioxide, carbon dioxide. Further, wet oxidation and pH-controlled hydrothermolysis are also considered chemical pre-treatment.

In a preferred embodiment the chemical pre-treatment is acid treatment, more preferably, a continuous dilute and/or mild acid treatment, such as, treatment with sulfuric acid, or another organic acid, such as acetic acid, citric acid, tartaric acid, succinic acid, hydrogen chloride or mixtures thereof. Other acids may also be used. Mild acid treatment means that the treatment pH lies in the range from 1-5, preferably pH 1-3. In a specific embodiment the acid concentration is in the range from 0.1 to 2.0 wt. % acid, preferably sulphuric acid. The acid may be contacted with the lignocellulose-containing material and the mixture may be held at a temperature in the range of 160-220° C., such as 165-195° C., for periods ranging from minutes to seconds, e.g., 1-60 minutes, such as 2-30 minutes or 3-12 minutes. Addition of strong acids, such as sulphuric acid, may be applied to remove hemicellulose. This enhances the digestibility of cellulose.

Other pre-treatment techniques are also contemplated according to the invention. Cellulose solvent treatment has been shown to convert about 90% of cellulose to glucose. It has also been shown that enzymatic hydrolysis could be greatly enhanced when the lignocellulose structure is disrupted. Alkaline H₂O₂, ozone, organosolv (uses Lewis acids, FeCl₃, (Al)₂SO₄ in aqueous alcohols), glycerol, dioxane, phenol, or ethylene glycol are among solvents known to disrupt cellulose structure and promote hydrolysis (Mosier et al., 2005, Bioresource Technology 96: 673-686).

Alkaline chemical pre-treatment with base, e.g., NaOH, Na₂CO₃ and/or ammonia or the like, is also within the scope of the invention. Pre-treatment methods using ammonia are described in, e.g., WO 2006/110891, WO 2006/110899, WO 2006/110900, WO 2006/110901, which are hereby incorporated by reference.

Wet oxidation techniques involve use of oxidizing agents, such as: sulphite based oxidizing agents or the like. Examples of solvent pre-treatments include treatment with DMSO (Dimethyl Sulfoxide) or the like. Chemical pre-treatment is generally carried out for 1 to 60 minutes, such as from 5 to 30 minutes, but may be carried out for shorter or longer periods of time dependent on the material to be pre-treated.

Other examples of suitable pre-treatment methods are described by Schell et al., 2003, Appl. Biochem and Biotechn. 105-108: 69-85, and Mosier et al., 2005, Bioresource Technology 96: 673-686, and US publication no. 2002/0164730, which references are hereby all incorporated by reference.

### Mechanical Pre-Treatment

The term “mechanical pre-treatment” refers to any mechanical (or physical) pre-treatment which promotes the separation and/or release of cellulose, hemicellulose and/or lignin from lignocellulose-containing material. For example, mechanical pre-treatment includes various types of milling, irradiation, steaming/steam explosion, and hydrothermolysis.

Mechanical pre-treatment includes comminution (mechanical reduction of the size). Comminution includes dry milling, wet milling and vibratory ball milling. Mechanical pre-treatment may involve high pressure and/or high temperature (steam explosion). In an embodiment of the invention high pressure means pressure in the range from 300 to 600 psi, preferably 400 to 500 psi, such as around 450 psi. In an embodiment of the invention high temperature means temperatures in the range from about 100 to 300° C., preferably from about 140 to 235° C. In a preferred embodiment mechanical pre-treatment is carried out as a batch-process, in a steam gun hydrolyzer system which uses high pressure and high temperature as defined above. A Sunds Hydrolyzer (available from Sunds Defibrator AB (Sweden) may be used for this.

### Combined Chemical and Mechanical Pre-Treatment

In a preferred embodiment the lignocellulose-containing material is subjected to both chemical and mechanical pre-treatment. For instance, the pre-treatment step may involve dilute or mild acid treatment and high temperature and/or pressure treatment. The chemical and mechanical pre-treatments may be carried out sequentially or simultaneously, as desired.

In a preferred embodiment the pre-treatment is carried out as a dilute and/or mild acid steam explosion step. In another preferred embodiment pre-treatment is carried out as an ammonia fiber explosion step (or AFEX pre-treatment step).

### Biological Pre-Treatment

The term “biological pre-treatment” refers to any biological pre-treatment which promotes the separation and/or release of cellulose, hemicellulose, and/or lignin from the lignocellulose-containing material. Known biological pre-treatment techniques involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh, P., and Singh, A., 1993, Physicochemical and biological treatments for enzymatic/microbial conversion of lignocellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of lignocellulosic hydrolysates for ethanol production, Enz. Microb. Tech. 18: 312-331; and Vallander, L., and Eriksson, K.-E. L., 1990, Production of ethanol from lignocellulosic materials: State of the art, Adv. Biochem. Eng./Biotechnol. 42: 63-95).

### Downstream Processing

The pre-treated lignocellulose-containing material treated according to a method of the invention may be subjected to downstream processing. In a preferred embodiment downstream processing is hydrolysis and/or fermentation to produce a fermentation product, such as ethanol. Hydrolysis and fermentation will be described further below.

### Hydrolysis

Before or simultaneously with fermenting pre-treated lignocellulose-containing material treated in accordance with a method of the invention the material is hydrolyzed enzymatically in order to break down especially cellulose and/or hemicellulose. Hydrolysis may be carried out as a fed batch process where the pre-treated lignocellulose-containing material treated in accordance with a method of the invention is fed continuously/gradually or stepwise to a vessel, tank, or the like, suitable for hydrolysis. Precisely how hydrolysis is carried out depends on which chemical(s) and/or enzyme(s) have already been added during treatment in accordance with a method of the invention. One skilled in the art can easily determine suitable hydrolysis conditions. The hydrolysis and/or fermentation steps are preferably carried out as SSF, HHF, or SHF.

Generally at least one or more cellulolytic enzyme(s) is(are) present during hydrolysis. If required further hydrolyzing enzymes and further additional chemicals may be added.

Generally enzyme(s) used for hydrolysis is(are) capable of directly or indirectly converting carbohydrate polymers into fermentable sugars which can be fermented into a desired fermentation product, such as ethanol.

In a preferred embodiment one or more hemicellulolytic enzymes are used for hydrolyzing the pre-treated lignocellulose-containing material. In one embodiment the hemicellulolytic enzyme(s) is(are) added in accordance with a method of the invention. However, it should be understood that there may be situations where it is advantageous to add one or more hydrolyzing enzymes after carrying out a treatment method of the invention.

Generally hemicellulose polymers are broken down by hemicellulolytic enzymes, such as hemicellulases to release its five and six carbon sugar components. The six carbon sugars (hexoses), such as glucose, galactose, arabinose, and mannose, can readily be fermented to fermentation products such as ethanol, acetone, butanol, glycerol, citric acid, fumaric acid etc. by suitable fermenting organisms including yeast.

In an embodiment the pre-treated lignocellulose-containing material is hydrolyzed in the presence of one or more hemicellulases, preferably selected from the group of xylanase, esterase, cellobiase, or combination thereof.

Hydrolysis may preferably be carried out in the presence of a combination of cellulase(s) and hemicellulase(s), and optionally one or more of the other enzyme activities mentioned above or in the “Enzyme” section below.

In a further embodiment xylose isomerase may be used while hydrolyzing pre-treated lignocellulose-containing material. Xylose isomerase can convert xylose to xylulose that can be fermented by fermenting organisms like Saccharomyces to a desired fermentation product. Consequently, in one embodiment xylose isomerise is added during treatment in accordance with a method of the invention or alternatively during hydrolysis (downstream processing).

Enzymatic treatment may be carried out in a suitable aqueous environment under conditions which can readily be determined by one skilled in the art. In a preferred embodiment hydrolysis is carried out at suitable, preferably optimal, conditions for the enzyme(s) in question.

Suitable process time, temperature and pH conditions can readily be determined by one skilled in the art. According to a preferred embodiment hydrolysis is carried out at a pH in the range from 4 to 8, preferably from 5 to 7. A suitable temperature during hydrolysis lies in the range from between 20 and 70° C., preferably between 25 and 60° C. Hydrolysis is typically carried out until the fermentable sugar yields are greater than 65%, preferably greater than 75%, more preferably greater than 85%. Typically hydrolysis is carried out for between 5 and 120 hours, preferable 16 to 96 hours, more preferably between 24 and 72 hours.

### Fermentation

According to the invention sugars from pre-treated and/or hydrolyzed lignocellulose-containing material are fermented using one or more fermenting organisms capable of fermenting fermentable sugars, such as glucose, xylose, mannose, and galactose directly or indirectly into a desired fermentation product. The fermentation conditions depend on the desired fermentation product and can easily be determined by one of ordinary skill in the art.

In the case of ethanol fermentation with yeast the fermentation is preferably ongoing for between 1-120 hours, preferably 5-96 hours. In an embodiment the fermentation is carried out at a temperature between 20 and 40° C., preferably between 26 and 34° C., in particular around 32° C. In an embodiment the pH is from pH 3-7, preferably 4-6.

In preferred embodiments fermentations are carried out separately from or simultaneous with hydrolysis. In preferred embodiments the hydrolysis and fermentation steps are carried out as SSF, HHF or SHF steps.

### Recovery

Subsequent to fermentation the fermentation product may optionally be separated from the fermentation medium in any suitable way. For instance, the medium may be distilled to extract the fermentation product or the fermentation product may be extracted from the fermentation medium by micro or membrane filtration techniques. Alternatively the fermentation product may be recovered by stripping. Recovery methods are well known in the art.

### Fermentation Products

The present invention may be used for producing any fermentation product. Preferred fermentation products include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B12, beta-carotene); and hormones.

Other products include consumable alcohol industry products, e.g., beer and wine; dairy industry products, e.g., fermented dairy products; leather industry products and tobacco industry products. In a preferred embodiment the fermentation product is an alcohol, especially ethanol. The fermentation product, such as ethanol, obtained according to the invention, may preferably be used as fuel alcohol/ethanol. However, in the case of ethanol it may also be used as potable ethanol.

### Fermenting Organisms

The term “fermenting organism” refers to any organism, including bacterial and fungal organisms, including yeast and filamentous fungi, suitable for producing a desired fermentation product. Especially suitable fermenting organisms according to the invention are able to ferment, i.e., convert, sugars, glucose, xylose, fructose and/or maltose, directly or indirectly into the desired fermentation product. Examples of fermenting organisms include fungal organisms, such as yeast. Preferred yeast includes strains of the genus Saccharomyces, in particular a strain of Saccharomyces cerevisiae or Saccharomyces uvarum; a strain of Pichia, in particular Pichia stipitis or Pichia pastoris; a strain of the genus Candida, in particular a strain of Candida utilis, Candida arabinofermentans, Candida diddensii, or Candida boidinii. Other contemplated yeast includes strains of Hansenula, in particular Hansenula polymorpha or Hansenula anomala; strains of Kluyveromyces in particular Kluyveromyces marxianus or Kluyveromyces fagilis, and strains of Schizosaccharomyces, in particular Schizosaccharomyces pombe.

Preferred bacterial fermenting organisms include strains of Escherichia, in particular Escherichia coli, strains of Zymomonas in particular Zymomonas mobilis, strains of Zymobacter in particular Zymobactor palmae, strains of Klebsiella in particular Klebsiella oxytoca, strains of Leuconostoc in particular Leuconostoc mesenteroides, strains of Clostridium in particular Clostridium butyricum, strains of Enterobacter in particular Enterobacter aerogenes and strains of Thermoanaerobacter, in particular Thermoanaerobacter BG1L1 (Appl. Micrbiol. Biotech. 77: 61-86) and Thermoanarobacter ethanolicus.

In an embodiment the fermenting organism(s) is(are) C6 sugar fermenting organisms, such as of a strain of, e.g., Saccharomyces cerevisiae.

In connection with especially fermentation of lignocellulose derived materials C5 sugar fermenting organisms are contemplated. Most C5 sugar fermenting organisms also ferment C6 sugars. Examples of C5 sugar fermenting organisms include strains of Pichia, such as of the species Pichia stipitis. C5 sugar fermenting bacteria are also known. Also some Saccharomyces cerevisae strains ferment C5 (and C6) sugars. Examples are genetically modified strains of Saccharomyces spp that are capable of fermenting C5 sugars include the ones concerned in, e.g., Ho et al., 1998, Applied and Environmental Microbiology, p. 1852-1859 and Karhumaa et al., 2006, Microbial Cell Factories 5:18.

In one embodiment the fermenting organism is added to the fermentation medium so that the viable fermenting organism, such as yeast, count per mL of fermentation medium is in the range from 10⁵ to 10¹², preferably from 10⁷ to 10¹⁰, especially about 5×10⁷.

Commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), FERMIOL (available from DSM Specialties), and a modified yeast from Royal Nedalco, NL.

### Lignocellulose-Containing Material

The term “lignocellulose-containing material” means material containing a significant content of cellulose, hemicellulose, and lignin. Lignocellulose-containing material is often referred to as “biomass”.

The lignocellulose-containing material may be any material containing lignocellulose. In a preferred embodiment the lignocellulose-containing material contains at least 30 wt. %, preferably at least 50 wt. %, more preferably at least 70 wt. %, even more preferably at least 90 wt. % lignocellulose. It is to be understood that lignocellulose-containing material may also comprise other constituents such as proteinaceous material, starchy material, sugars, such as fermentable sugars and/or un-fermentable sugars.

Lignocellulose-containing material is generally found, for example, in the stems, leaves, hulls, husks, and cobs of plants or leaves, branches, and wood of trees. Lignocellulose-containing material can also be, but is not limited to, herbaceous material, agricultural residues, forestry residues, municipal solid wastes, waste paper, and pulp and paper mill residues. It is to be understood that lignocellulose-containing material may be in the form of plant cell wall material containing lignin, cellulose and hemicellulose in a mixed matrix.

In a preferred embodiment the lignocellulose-containing material is corn cobs, corn fiber, rice straw, pine wood, wood chips, poplar, bagasse, paper and pulp processing waste.

Other examples include corn stover, hardwood such as poplar and birch, softwood, cereal straw such as wheat straw, switchgrass, Miscanthus, rice hulls, municipal solid waste (MSW), industrial organic waste, office paper, or mixtures thereof.

In a preferred embodiment the lignocellulose-containing material is corn stover or corn cobs. In another preferred embodiment, the lignocellulose-containing material is corn fiber. In another preferred embodiment, the lignocellulose-containing material is switch grass. In another preferred embodiment, the lignocellulose-containing material is bagasse.

### Enzymes

Even if not specifically mentioned in context of a method or process of the invention, it is to be understood that the enzyme(s) (as well as other compounds) are used in an effective amount.

### Hydrolyzing Enzymes

According to the invention hydrolyzing enzymes include cellulolytic enzymes, hemicellulytic enzymes, esterase enzymes, amylolytic enzymes and pectolytic enzymes, proteolytic enzymes, including those listed below.

### Cellulolytic Activity

The term “cellulolytic activity” as used herein are understood as comprising enzymes having cellobiohydrolase activity (EC 3.2.1.91), e.g., cellobiohydrolase I and cellobiohydrolase II, as well as endo-glucanase activity (EC 3.2.1.4) and beta-glucosidase activity (EC 3.2.1.21).

In order to be efficient, the digestion of cellulose may require several types of enzymes acting cooperatively. At least three categories of enzymes are often needed to convert cellulose into glucose: endoglucanases (EC 3.2.1.4) that cut the cellulose chains at random; cellobiohydrolases (EC 3.2.1.91) which cleave cellobiosyl units from the cellulose chain ends and beta-glucosidases (EC 3.2.1.21) that convert cellobiose and soluble cellodextrins into glucose. Among these three categories of enzymes involved in the biodegradation of cellulose, cellobiohydrolases are the key enzymes for the degradation of native crystalline cellulose. The term “cellobiohydrolase I” is defined herein as a cellulose 1,4-beta-cellobiosidase (also referred to as Exo-glucanase, Exo-cellobiohydrolase or 1,4-beta-cellobiohydrolase) activity, as defined in the enzyme class EC 3.2.1.91, which catalyzes the hydrolysis of 1,4-beta-D-glucosidic linkages in cellulose and cellotetraose, by the release of cellobiose from the non-reducing ends of the chains. The definition of the term “cellobiohydrolase II activity” is identical, except that cellobiohydrolase II attacks from the reducing ends of the chains.

The cellulases may comprise a carbohydrate-binding module (CBM) which enhances the binding of the enzyme to a cellulose-containing fiber and increases the efficacy of the catalytic active part of the enzyme. A CBM is defined as contiguous amino acid sequence within a carbohydrate-active enzyme with a discreet fold having carbohydrate-binding activity. For further information of CBMs see the CAZy internet server (Supra) or Tomme et al. (1995) in Enzymatic Degradation of Insoluble Polysaccharides (Saddler and Penner, eds.), Cellulose-binding domains: classification and properties. pp. 142-163, American Chemical Society, Washington.

The cellulolytic activity may, in a preferred embodiment, be in the form of a preparation of enzymes of fungal origin, such as from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense (see e.g., US publication #2007/0238155 from Dyadic Inc, USA).

In preferred embodiment the cellulolytic enzyme preparation contains one or more of the following activities: cellulase, hemicellulase, cellulolytic enzyme enhancing activity, beta-glucosidase activity, endoglucanase, cellubiohydrolase, or xylose-isomerase.

In a preferred embodiment the cellulases or cellulolytic enzymes may be a cellulolytic preparation as defined PCT/2008/065417, which is hereby incorporated by reference. In a preferred embodiment the cellulolytic preparation comprising a polypeptide having cellulolytic enhancing activity (GH61A), preferably the one disclosed in WO 2005/074656. The cellulolytic preparation may further comprise a beta-glucosidase, such as a beta-glucosidase derived from a strain of the genus Trichoderma, Aspergillus or Penicillium, including the fusion protein having beta-glucosidase activity disclosed in WO2008/057637 (Novozymes). In an embodiment the cellulolytic preparation may also comprises a CBH II, preferably Thielavia terrestris cellobiohydrolase II (CEL6A). In another preferred embodiment the cellulolytic enzyme preparation may also comprise cellulolytic enzymes, preferably one derived from Trichoderma reesei, Humicola insolens and/or Chrysosporium lucknowense.

In an embodiment the cellulolytic enzyme preparation comprises a polypeptide having cellulolytic enhancing activity (GH61A) disclosed in WO 2005/074656; a cellobiohydrolase, such as Thielavia terrestris cellobiohydrolase II (CEL6A), a beta-glucosidase (e.g., the fusion protein disclosed in WO 2008/057634) and cellulolytic enzymes, e.g., derived from Trichoderma reesei.

In an embodiment the cellulolytic enzyme preparation comprises a polypeptide having cellulolytic enhancing activity (GH61A) disclosed in WO 2005/074656; a beta-glucosidase (e.g., the fusion protein disclosed in WO 2008/057637) and cellulolytic enzymes, e.g., derived from Trichoderma reesei.

In an embodiment the cellulolytic enzyme composition is the commercially available product CELLUCLAST™ 1.5 L, CELLUZYME™ (from Novozymes A/S, Denmark) or ACCELERASE™ 1000 (from Genencor Inc. USA).

A cellulase may be added for hydrolyzing the pre-treated lignocellulose-containing material. The cellulase may be dosed in the range from 0.1-100 FPU per gram total solids (TS), preferably 0.5-50 FPU per gram TS, especially 1-20 FPU per gram TS. In another embodiment at least 0.1 mg cellulolytic enzyme per gram total solids (TS), preferably at least 3 mg cellulolytic enzyme per gram TS, such as between 5 and 10 mg cellulolytic enzyme(s) per gram TS is(are) used for hydrolysis.

### Endoglucanase (EG)

The term “endoglucanase” means an endo-1,4-(1,3;1,4)-beta-D-glucan 4-glucanohydrolase (E.C. No. 3.2.1.4), which catalyzes endo-hydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity may be determined using carboxymethyl cellulose (CMC) hydrolysis according to the procedure of Ghose, 1987, Pure and Appl. Chem. 59: 257-268.

In a preferred embodiment endoglucanases may be derived from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

### Cellobiohydrolase (CBH)

The term “cellobiohydrolase” means a 1,4-beta-D-glucan cellobiohydrolase (E.C. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain.

Examples of cellobiohydroloses are mentioned above including CBH I and CBH II from Trichoderma reseei; Humicola insolens and CBH II from Thielavia terrestris cellobiohydrolase (CEL6A).

Cellobiohydrolase activity may be determined according to the procedures described by Lever et al., 1972, Anal. Biochem. 47: 273-279 and by van Tilbeurgh et al., 1982, FEBS Letters 149: 152-156; van Tilbeurgh and Claeyssens, 1985, FEBS Letters 187: 283-288. The Lever et al. method is suitable for assessing hydrolysis of cellulose in corn stover and the method of van Tilbeurgh et al. is suitable for determining the cellobiohydrolase activity on a fluorescent disaccharide derivative.

### Beta-Glucosidase

One or more beta-glucosidases may be present during hydrolysis.

The term “beta-glucosidase” means a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi et al., 2002, J. Basic Microbiol. 42: 55-66, except different conditions were employed as described herein. One unit of beta-glucosidase activity is defined as 1.0 μmole of p-nitrophenol produced per minute at 50° C., pH 5 from 4 mM p-nitrophenyl-beta-D-glucopyranoside as substrate in 100 mM sodium citrate, 0.01% TWEEN® 20.

In a preferred embodiment the beta-glucosidase is of fungal origin, such as a strain of the genus Trichoderma, Aspergillus or Penicillium. In a preferred embodiment the beta-glucosidase is a derived from Trichoderma reesei, such as the beta-glucosidase encoded by the bgl1 gene (see FIG. 1 of EP 562003). In another preferred embodiment the beta-glucosidase is derived from Aspergillus oryzae (recombinantly produced in Aspergillus oryzae according to WO 02/095014), Aspergillus fumigatus (recombinantly produced in Aspergillus oryzae according to Example 22 of WO 02/095014) or Aspergillus niger (1981, J. Appl. 3: 157-163). In a preferred embodiment the beta-glucosidase is a fusion protein disclosed in U.S. 60/832,511 or PCT/US2007/074038 (Novozymes Inc, USA) such as the Humicola insolens endoglucanase V core domain fused to Aspergillus oryzae beta-glucosidase.

### Hemicellulolytic Enzymes

According to the invention the pre-treated lignocellulose-containing material may further be subjected to one or more hemicellulolytic enzymes, e.g., one or more hemicellulases.

Hemicellulose can be broken down by hemicellulases and/or acid hydrolysis to release its five and six carbon sugar components.

In an embodiment of the invention the lignocellulose derived material may be treated with one or more hemicellulases.

Any hemicellulase suitable for use in hydrolyzing hemicellulose, preferably into xylose, may be used. Preferred hemicellulases include xylanases, arabinofuranosidases, acetyl xylan esterase, feruloyl esterase, glucuronidases, endo-galactanase, mannases, endo or exo arabinases, exo-galactanses, and mixtures of two or more thereof. Preferably, the hemicellulase for use in the present invention is an endo-acting hemicellulase, and more preferably, the hemicellulase is an endo-acting hemicellulase which has the ability to hydrolyze hemicellulose under acidic conditions of below pH 7, preferably pH 3-7.

An example of hemicellulase suitable for use in the present invention includes VISCOZYME™ (available from Novozymes A/S, Denmark).

In an embodiment the hemicellulase is a xylanase. In an embodiment the xylanase may preferably be of microbial origin, such as of fungal origin (e.g., Trichoderma, Meripilus, Humicola, Aspergillus, Fusarium) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME™ and BIOFEED WHEAT™ from Novozymes A/S, Denmark.

The hemicellulase may be added in an amount effective to hydrolyze hemicellulose, such as, in amounts from about 0.001 to 0.5 wt. %, more preferably from about 0.05 to 0.5 wt. % of insoluble solids.

Xylanases may be added to step a) and/or step a′) or step e) in amounts of 0.001-1.0 g/kg insoluble solids, preferably in an amount of 0.005-0.5 g/kg insoluble solids, and most preferably from 0.05-0.10 g/kg insoluble solids.

### Cellulolytic Enhancing Activity

The term “cellulolytic enhancing activity” is defined herein as a biological activity that enhances the hydrolysis of a lignocellulose derived material by proteins having cellulolytic activity. For purposes of the present invention, cellulolytic enhancing activity is determined by measuring the increase in reducing sugars or in the increase of the total of cellobiose and glucose from the hydrolysis of a lignocellulose derived material, e.g., pre-treated lignocellulose-containing material by cellulolytic protein under the following conditions: 1-50 mg of total protein/g of cellulose in PCS (pre-treated corn stover), wherein total protein is comprised of 80-99.5% w/w cellulolytic protein/g of cellulose in PCS and 0.5-20% w/w protein of cellulolytic enhancing activity for 1-7 day at 50° C. compared to a control hydrolysis with equal total protein loading without cellulolytic enhancing activity (1-50 mg of cellulolytic protein/g of cellulose in PCS).

The polypeptides having cellulolytic enhancing activity enhance the hydrolysis of a lignocellulose derived material catalyzed by proteins having cellulolytic activity by reducing the amount of cellulolytic enzyme required to reach the same degree of hydrolysis preferably at least 0.1-fold, more at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

In a preferred embodiment the hydrolysis and/or fermentation is carried out in the presence of a cellulolytic enzyme in combination with a polypeptide having enhancing activity. In a preferred embodiment the polypeptide having enhancing activity is a family GH61A polypeptide. WO 2005/074647 discloses isolated polypeptides having cellulolytic enhancing activity and polynucleotides thereof from Thielavia terrestris. WO 2005/074656 discloses an isolated polypeptide having cellulolytic enhancing activity and a polynucleotide thereof from Thermoascus aurantiacus. U.S. Published Application No. 2007/0077630 discloses an isolated polypeptide having cellulolytic enhancing activity and a polynucleotide thereof from Trichoderma reesei.

### Xylose Isomerase

Xylose isomerases (D-xylose ketoisomerase) (E.C. 5.3.1.5.) are enzymes that catalyze the reversible isomerization reaction of D-xylose to D-xylulose. Some xylose isomerases also convert the reversible isomerization of D-glucose to D-fructose. Therefore, xylose isomarase is sometimes referred to as “glucose isomerase”.

A xylose isomerase used in a method or process of the invention may be any enzyme having xylose isomerase activity and may be derived from any sources, preferably bacterial or fungal origin, such as filamentous fungi or yeast. Examples of bacterial xylose isomerases include the ones belonging to the genera Streptomyces, Actinoplanes, Bacillus and Flavobacterium, and Thermotoga, including T. neapolitana (Vieille et al., 1995, Appl. Environ. Microbiol. 61(5): 1867-1875) and T. maritime.

Examples of fungal xylose isomerases are derived species of Basidiomycetes.

A preferred xylose isomerase is derived from a strain of yeast genus Candida, preferably a strain of Candida boidinii, especially the Candida boidinii xylose isomerase disclosed by, e.g., Vongsuvanlert et al., 1988, Agric. Biol. Chem., 52(7): 1817-1824. The xylose isomerase may preferably be derived from a strain of Candida boidinii (Kloeckera 2201), deposited as DSM 70034 and ATCC 48180, disclosed in Ogata et al., Agric. Biol. Chem. 33: 1519-1520 or Vongsuvanlert et al., 1988, Agric. Biol. Chem. 52(2): 1519-1520.

In one embodiment the xylose isomerase is derived from a strain of Streptomyces, e.g., derived from a strain of Streptomyces murinus (U.S. Pat. No. 4,687,742); S. flavovirens, S. albus, S. achromogenus, S. echinatus, S. wedmorensis all disclosed in U.S. Pat. No. 3,616,221. Other xylose isomerases are disclosed in U.S. Pat. No. 3,622,463, U.S. Pat. No. 4,351,903, U.S. Pat. No. 4,137,126, U.S. Pat. No. 3,625,828, HU patent no. 12,415, DE patent 2,417,642, JP patent no. 69,28,473, and WO 2004/044129 (which as all incorporated by reference.

The xylose isomerase may be either in immobilized or liquid form. Liquid form is preferred.

The xylose isomerase is added to provide an activity level in the range from 0.01-100 IGIU per gram insoluble solids.

Examples of commercially available xylose isomerases include SWEETZYME™ T from Novozymes.

### Alpha-Amylase

According to the invention any alpha-amylase may be used, such as of fungal, bacterial or plant origin. In a preferred embodiment the alpha-amylase is an acid alpha-amylase, e.g., acid fungal alpha-amylase or acid bacterial alpha-amylase. The term “acid alpha-amylase” means an alpha-amylase (E.C. 3.2.1.1) which added in an effective amount has activity optimum at a pH in the range of 3 to 7, preferably from 3.5 to 6, or more preferably from 4-5.

### Bacterial Alpha-Amylase

According to the invention a bacterial alpha-amylase is preferably derived from the genus Bacillus.

In a preferred embodiment the Bacillus alpha-amylase is derived from a strain of Bacillus lichenifonnis, Bacillus amyloliquefaciens, Bacillus subtilis or Bacillus stearothermophilus, but may also be derived from other Bacillus sp. Specific examples of contemplated alpha-amylases include the Bacillus lichenifonnis alpha-amylase shown in SEQ ID NO: 4 in WO 99/19467, the Bacillus amyloliquefaciens alpha-amylase SEQ ID NO: 5 in WO 99/19467 and the Bacillus stearothermophilus alpha-amylase shown in SEQ ID NO: 3 in WO 99/19467 (all sequences hereby incorporated by reference). In an embodiment the alpha-amylase may be an enzyme having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NOS: 1, 2 or 3, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or U.S. Pat. No. 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta (181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta (181-182) and further comprise a N193F substitution (also denoted I181*+G182*+N193F) compared to the wild-type BSG alpha-amylase amino acid sequence set forth in SEQ ID NO:3 disclosed in WO 99/19467.

### Bacterial Hybrid Alpha-Amylase

A hybrid alpha-amylase specifically contemplated comprises 445 C-terminal amino acid residues of the Bacillus lichenifonnis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467), with one or more, especially all, of the following substitution:

G48A+T49I+G107A+H156Y+A181T+N190F+I201F+A209V+Q264S (using the Bacillus lichenifonnis numbering in SEQ ID NO: 4 of WO 99/19467). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 99/19467).

In an embodiment the bacterial alpha-amylase is dosed in an amount of 0.0005-5 KNU per g DS, preferably 0.001-1 KNU per g DS, such as around 0.050 KNU per g DS.

### Fungal Alpha-Amylase

Fungal alpha-amylases include alpha-amylases derived from a strain of the genus Aspergillus, such as, Aspergillus oryzae, Aspergillus niger and Aspergillis kawachii alpha-amylases.

A preferred acidic fungal alpha-amylase is a Fungamyl-like alpha-amylase which is derived from a strain of Aspergillus oryzae. According to the present invention, the term “Fungamyl-like alpha-amylase” indicates an alpha-amylase which exhibits a high identity, i.e., at least 70%, at least 75%, at least 80%, at least 85%, at least 90%, at least 95%, at least 96%, at least 97%, at least 98%, at least 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from Aspergillus niger disclosed as “AMYA_ASPNG” in the Swiss-prot/TeEMBL database under the primary accession no. P56271 and described in WO 89/01969 (Example 3—incorporated by reference). A commercially available acid fungal alpha-amylase derived from Aspergillus niger is SP288 (available from Novozymes A/S, Denmark).

Other contemplated wild-type alpha-amylases include those derived from a strain of the genera Rhizomucor and Meripilus, preferably a strain of Rhizomucor pusillus (WO 2004/055178 incorporated by reference) or Meripilus giganteus.

In a preferred embodiment the alpha-amylase is derived from Aspergillus kawachii and disclosed by Kaneko et al., 1996, J. Ferment. Bioeng. 81:292-298, “Molecular-cloning and determination of the nucleotide-sequence of a gene encoding an acid-stable alpha-amylase from Aspergillus kawachii”; and further as EMBL:#AB008370.

The fungal alpha-amylase may also be a wild-type enzyme comprising a starch-binding domain (SBD) and an alpha-amylase catalytic domain (i.e., none-hybrid), or a variant thereof. In an embodiment the wild-type alpha-amylase is derived from a strain of Aspergillus kawachii.

### Fungal Hybrid Alpha-Amylase

In a preferred embodiment the fungal acid alpha-amylase is a hybrid alpha-amylase. Preferred examples of fungal hybrid alpha-amylases include the ones disclosed in WO 2005/003311 or U.S. Patent Publication no. 2005/0054071 (Novozymes) or U.S. patent application No. 60/638,614 (Novozymes) which is hereby incorporated by reference. A hybrid alpha-amylase may comprise an alpha-amylase catalytic domain (CD) and a carbohydrate-binding domain/module (CBM), such as a starch binding domain, and optional a linker.

Specific examples of contemplated hybrid alpha-amylases include those disclosed in Table 1 to 5 of the examples in U.S. patent application No. 60/638,614, including Fungamyl variant with catalytic domain JA118 and Athelia rolfsii SBD (SEQ ID NO:100 in U.S. 60/638,614), Rhizomucor pusillus alpha-amylase with Athelia rolfsii AMG linker and SBD (SEQ ID NO:101 in U.S. 60/638,614), Rhizomucor pusillus alpha-amylase with Aspergillus niger glucoamylase linker and SBD (which is disclosed in Table 5 as a combination of amino acid sequences SEQ ID NO:20, SEQ ID NO:72 and SEQ ID NO:96 in U.S. application Ser. No. 11/316,535) or as V039 in Table 5 in WO 2006/069290, and Meripilus giganteus alpha-amylase with Athelia rolfsii glucoamylase linker and SBD (SEQ ID NO:102 in U.S. 60/638,614). Other specifically contemplated hybrid alpha-amylases are any of the ones listed in Tables 3, 4, 5, and 6 in Example 4 in U.S. application Ser. No. 11/316,535 and WO 2006/069290 (hereby incorporated by reference).

Other specific examples of contemplated hybrid alpha-amylases include those disclosed in U.S. Patent Publication no. 2005/0054071, including those disclosed in Table 3 on page 15, such as Aspergillus niger alpha-amylase with Aspergillus kawachii linker and starch binding domain.

Contemplated are also alpha-amylases which exhibit a high identity to any of above mention alpha-amylases, i.e., at least 70%, at least 75%, at least 80%, at least 85%, at least 90%, at least 95%, at least 96%, at least 97%, at least 98%, at least 99% or even 100% identity to the mature enzyme sequences.

An acid alpha-amylase may according to the invention be added in an amount of 0.001 to 10 AFAU/g insoluble solids, preferably from 0.01 to 5 AFAU/g insoluble solids, especially 0.3 to 2 AFAU/g insoluble solids or 0.001 to 1 FAU-F/g insoluble solids, preferably 0.01 to 1 FAU-F/g insoluble solids.

### Commercial Alpha-Amylase Products

Preferred commercial compositions comprising alpha-amylase include MYCOLASE™ from DSM (Gist Brocades), BAN™, TERMAMYL™ SC, FUNGAMYL™, LIQUOZYME™ X, LIQUOZYME™ SC and SAN™ SUPER, SAN™ EXTRA L (Novozymes A/S) and CLARASE™ L-40,000, DEX-LO™, SPEZYME™ FRED, SPEZYME™ AA, and SPEZYME™ DELTA AA (Genencor Int.), and the acid fungal alpha-amylase sold under the trade name SP288 (available from Novozymes A/S, Denmark).

### Carbohydrate-Source Generating Enzyme

The term “carbohydrate-source generating enzyme” includes glucoamylase (being glucose generators), beta-amylase and maltogenic amylase (being maltose generators) and also pullulanase and alpha-glucosidase. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated mixtures are mixtures of at least a glucoamylase and an alpha-amylase, especially an acid amylase, even more preferred an acid fungal alpha-amylase. The ratio between acid fungal alpha-amylase activity (FAU-F) and glucoamylase activity (AGU) (i.e., FAU-F per AGU) may in an embodiment of the invention be between 0.1 and 100, in particular between 2 and 50, such as in the range from 10-40.

### Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al., 1984, EMBO J. 3(5): 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem., 1991, 55(4): 941-949), or variants or fragments thereof. Other Aspergillus glucoamylase variants include variants with enhanced thermal stability: G137A and G139A (Chen et al., 1996, Prot. Eng. 9: 499-505); D257E and D293E/Q (Chen et al., 1995, Prot. Eng. 8, 575-582); N182 (Chen et al., 1994, Biochem. J. 301: 275-281); disulphide bonds, A246C (Fierobe et al., 1996, Biochemistry 35: 8698-8704; and introduction of Pro residues in position A435 and S436 (Li et al., 1997, Protein Eng. 10: 1199-1204.

Other glucoamylases include Athelia rolfsii (previously denoted Corticium rolfsii) glucoamylase (see U.S. Pat. No. 4,727,026 and (Nagasaka et al., 1998, “Purification and properties of the raw-starch-degrading glucoamylases from Corticium rolfsii, Appl Microbiol Biotechnol. 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamylolyticum (EP 135,138), and C. thermohydrosulfuricum (WO 86/01831) and Trametes cingulata, Pachykytospora papyracea; and Leucopaxillus giganteus all disclosed in WO 2006/069289; or Peniophora rufomarginata disclosed in PCT/US2007/066618; or a mixture thereof. Also hybrid glucoamylase are contemplated according to the invention. Examples the hybrid glucoamylases disclosed in WO 2005/045018. Specific examples include the hybrid glucoamylase disclosed in Tables 1 and 4 of Example 1 (which hybrids are hereby incorporated by reference).

Contemplated are also glucoamylases which exhibit a high identity to any of above mention glucoamylases, i.e., at least 70%, at least 75%, at least 80%, at least 85%, at least 90%, at least 95%, at least 96%, at least 97%, at least 98%, at least 99% or even 100% identity to the mature enzymes sequences mentioned above.

Commercially available compositions comprising glucoamylase include AMG 200 L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.0001-20 AGU/g DS, preferably 0.001-10 AGU/g insoluble solids, especially between 0.01-5 AGU/g insoluble solids, such as 0.1-2 AGU/g insoluble solids.

### Beta-Amylase

A beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (Fogarty and Kelly, 1979, Progress in Industrial Microbiology 15: 112-115). These beta-amylases are characterized by having optimum temperatures in the range from 40° C. to 65° C. and optimum pH in the range from 4.5 to 7. A commercially available beta-amylase from barley is NOVOZYM™ WBA from Novozymes A/S, Denmark and SPEZYME™ BBA 1500 from Genencor Int., USA.

### Maltogenic Amylase

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference.

The maltogenic amylase may in a preferred embodiment be added in an amount of 0.05-5 mg total protein/gram insoluble solids or 0.05-5 MANU/g insoluble solids.

### Proteases

A protease may be added during hydrolysis in step ii), fermentation in step iii) or simultaneous hydrolysis and fermentation. The protease may be any protease. In a preferred embodiment the protease is an acid protease of microbial origin, preferably of fungal or bacterial origin. An acid fungal protease is preferred, but also other proteases can be used.

Suitable proteases include microbial proteases, such as fungal and bacterial proteases. Preferred proteases are acidic proteases, i.e., proteases characterized by the ability to hydrolyze proteins under acidic conditions below pH 7.

Contemplated acid fungal proteases include fungal proteases derived from Aspergillus, Mucor, Rhizopus, Candida, Coriolus, Endothia, Enthomophtra, Irpex, Penicillium, Sclerotiumand Torulopsis. Especially contemplated are proteases derived from Aspergillus niger (see, e.g., Koaze et al., 1964, Agr. Biol. Chem. Japan 28: 216), Aspergillus saitoi (see, e.g., Yoshida, 1954, J. Agr. Chem. Soc. Japan 28: 66), Aspergillus awamori (Hayashida et al., 1977, Agric. Biol. Chem. 42(5): 927-933, Aspergillus aculeatus (WO 95/02044), or Aspergillus oryzae, such as the pepA protease; and acidic proteases from Mucor pusillus or Mucor miehei.

Contemplated are also neutral or alkaline proteases, such as a protease derived from a strain of Bacillus. A particular protease contemplated for the invention is derived from Bacillus amyloliquefaciens and has the sequence obtainable at Swissprot as Accession No. PO6832. Also contemplated are the proteases having at least 90% identity to amino acid sequence obtainable at Swissprot as Accession No. PO6832 such as at least 92%, at least 95%, at least 96%, at least 97%, at least 98%, or particularly at least 99% identity.

Further contemplated are the proteases having at least 90% identity to amino acid sequence disclosed as SEQ.ID.NO:1 in the WO 2003/048353 such as at 92%, at least 95%, at least 96%, at least 97%, at least 98%, or particularly at least 99% identity.

Also contemplated are papain-like proteases such as proteases within E.C. 3.4.22.* (cysteine protease), such as EC 3.4.22.2 (papain), EC 3.4.22.6 (chymopapain), EC 3.4.22.7 (asclepain), EC 3.4.22.14 (actimidain), EC 3.4.22.15 (cathepsin L), EC 3.4.22.25 (glycyl endopeptidase) and EC 3.4.22.30 (caricain).

In an embodiment the protease is a protease preparation derived from a strain of Aspergillus, such as Aspergillus oryzae. In another embodiment the protease is derived from a strain of Rhizomucor, preferably Rhizomucor mehei. In another contemplated embodiment the protease is a protease preparation, preferably a mixture of a proteolytic preparation derived from a strain of Aspergillus, such as Aspergillus oryzae, and a protease derived from a strain of Rhizomucor, preferably Rhizomucor mehei.

Aspartic acid proteases are described in, for example, Handbook of Proteolytic Enzymes, Edited by A. J. Barrett, N. D. Rawlings and J. F. Woessner, Academic Press, San Diego, 1998, Chapter 270). Suitable examples of aspartic acid protease include, e.g., those disclosed in. Berka et al., 1990, Gene 96: 313); (Berka et al., 1993, Gene 125: 195-198); and Gomi et al., 1993, Biosci. Biotech. Biochem. 57: 1095-1100, which are hereby incorporated by reference.

Commercially available products include ALCALASE®, ESPERASE™ FLAVOURZYME™, PROMIX™, NEUTRASE®, RENNILASE®, NOVOZYM™ FM 2.0 L, and NOVOZYM™ 50006 (available from Novozymes A/S, Denmark) and GC106™ and SPEZYME™ FAN from Genencor Int., Inc., USA.

The protease may be present in an amount of 0.0001-1 mg enzyme protein per g insoluble solids, preferably 0.001 to 0.1 mg enzyme protein per g insoluble solids. Alternatively, the protease may be present in an amount of 0.0001 to 1 LAPU/g insoluble solids, preferably 0.001 to 0.1 LAPU/g insoluble solids and/or 0.0001 to 1 mAU-RH/g insoluble solids, preferably 0.001 to 0.1 mAU-RH/g insoluble solids.

### Materials & Methods

### Enzymes:

Cellulolytic preparation A: Cellulolytic composition comprising a polypeptide having cellulolytic enhancing activity (GH61A) disclosed in WO 2005/074656; a beta-glucosidase (fusion protein disclosed in WO2008/057637); and cellulolytic enzymes preparation derived from Trichoderma reesei. Cellulase preparation A is disclosed in co-pending U.S. application Ser. No. 12/130,838.
Yeast: RED START™ available from Red Star/Lesaffre, USA
Pre-treated corn stover used in Example 1 is dilute acid-catalyzed steam explosion corn stover (29.5 wt. % DS—batch 1752-91) obtained from NREL (National Renewable Research Laboratory, USA).

### Methods

### Determination of Identity

The relatedness between two amino acid sequences or between two nucleotide sequences is described by the parameter “identity”.

The degree of identity between two amino acid sequences may be determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=1, gap penalty=3, windows=5, and diagonals=5.

The degree of identity between two nucleotide sequences may be determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

### Measurement of Cellulase Activity Using Filter Paper Assay (FPU Assay)

### 1. Source of Method

1.1 The method is disclosed in a document entitled “Measurement of Cellulase Activities” by Adney, B. and Baker, J. 1996. Laboratory Analytical Procedure, LAP-006, National Renewable Energy Laboratory (NREL). It is based on the IUPAC method for measuring cellulase activity (Ghose, T. K., Measurement of Cellulse Activities, Pure & Appl. Chem. 59, pp. 257-268, 1987.

### 2. Procedure

2.1 The method is carried out as described by Adney and Baker, 1996, supra, except for the use of a 96 well plates to read the absorbance values after color development, as described below.

### 2.2 Enzyme Assay Tubes:

2.2.1 A rolled filter paper strip (#1 Whatman; 1×6 cm; 50 mg) is added to the bottom of a test tube (13×100 mm).
2.2.2 To the tube is added 1.0 mL of 0.05 M Na-citrate buffer (pH 4.80).
2.2.3 The tubes containing filter paper and buffer are incubated 5 min. at 50° C. (±0.1° C.) in a circulating water bath.
2.2.4 Following incubation, 0.5 mL of enzyme dilution in citrate buffer is added to the tube. Enzyme dilutions are designed to produce values slightly above and below the target value of 2.0 mg glucose.
2.2.5 The tube contents are mixed by gently vortexing for 3 seconds.
2.2.6 After vortexing, the tubes are incubated for 60 mins. at 50° C. (±0.1° C.) in a circulating water bath.
2.2.7 Immediately following the 60 min. incubation, the tubes are removed from the water bath, and 3.0 mL of DNS reagent is added to each tube to stop the reaction. The tubes are vortexed 3 seconds to mix.

### 2.3 Blank and Controls

2.3.1 A reagent blank is prepared by adding 1.5 mL of citrate buffer to a test tube.
2.3.2 A substrate control is prepared by placing a rolled filter paper strip into the bottom of a test tube, and adding 1.5 mL of citrate buffer.
2.3.3 Enzyme controls are prepared for each enzyme dilution by mixing 1.0 mL of citrate buffer with 0.5 mL of the appropriate enzyme dilution.
2.3.4 The reagent blank, substrate control, and enzyme controls are assayed in the same manner as the enzyme assay tubes, and done along with them.

### 2.4 Glucose Standards

2.4.1 A 100 mL stock solution of glucose (10.0 mg/mL) is prepared, and 5 mL aliquots are frozen. Prior to use, aliquots are thawed and vortexed to mix.
2.4.2 Dilutions of the stock solution are made in citrate buffer as follows:

G1=1.0 mL stock+0.5 mL buffer=6.7 mg/mL=3.3 mg/0.5 mL

G2=0.75 mL stock+0.75 mL buffer=5.0 mg/mL=2.5 mg/0.5 mL

G3=0.5 mL stock+1.0 mL buffer=3.3 mg/mL=1.7 mg/0.5 mL

G4=0.2 mL stock+0.8 mL buffer=2.0 mg/mL=1.0 mg/0.5 mL
2.4.3 Glucose standard tubes are prepared by adding 0.5 mL of each dilution to 1.0 mL of citrate buffer.
2.4.4 The glucose standard tubes are assayed in the same manner as the enzyme assay tubes, and done along with them.

### 2.5 Color Development

2.5.1 Following the 60 min. incubation and addition of DNS, the tubes are all boiled together for 5 mins. in a water bath.
2.5.2 After boiling, they are immediately cooled in an ice/water bath.
2.5.3 When cool, the tubes are briefly vortexed, and the pulp is allowed to settle. Then each tube is diluted by adding 50 microL from the tube to 200 microL of ddH2O in a 96-well plate. Each well is mixed, and the absorbance is read at 540 nm.

### 2.6 Calculations (Examples are Given in the NREL Document)

2.6.1 A glucose standard curve is prepared by graphing glucose concentration (mg/0.5 mL) for the four standards (G1-G4) vs. A₅₄₀. This is fitted using a linear regression (Prism Software), and the equation for the line is used to determine the glucose produced for each of the enzyme assay tubes.
2.6.2 A plot of glucose produced (mg/0.5 mL) vs. total enzyme dilution is prepared, with the Y-axis (enzyme dilution) being on a log scale.
2.6.3 A line is drawn between the enzyme dilution that produced just above 2.0 mg glucose and the dilution that produced just below that. From this line, it is determined the enzyme dilution that would have produced exactly 2.0 mg of glucose.
2.6.4 The Filter Paper Units/mL (FPU/mL) are calculated as follows:

FPU/mL=0.37/enzyme dilution producing 2.0 mg glucose

### Glucoamylase Activity

Glucoamylase activity may be measured in Glucoamylase Units (AGU).

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | |
|---|---|
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |

| Color reaction: | |
|---|---|
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Acid Alpha-Amylase Activity

When used according to the present invention the activity of an acid alpha-amylase may be measured in AFAU (Acid Fungal Alpha-amylase Units) or FAU-F.

### Acid Alpha-Amylase Activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

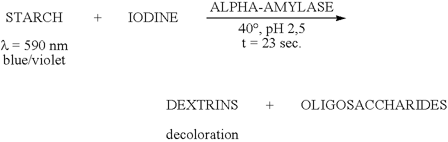

Standard conditions/reaction conditions:
Substrate: Soluble starch, approx. 0.17 g/L
Buffer: Citrate, approx. 0.03 M
Iodine (12): 0.03 g/L
CaCl₂: 1.85 mM
pH: 2.50±0.05
Incubation temperature: 40° C.
Reaction time: 23 seconds
Wavelength: 590 nm
Enzyme concentration: 0.025 AFAU/mL
Enzyme working range: 0.01-0.04 AFAU/mL

A folder EB-SM-0259.02/01 describing this analytical method in more detail is available upon request to Novozymes A/S, Denmark, which folder is hereby included by reference.

### Determination of FAU-F

FAU-F Fungal Alpha-Amylase Units (Fungamyl) is measured relative to an enzyme standard of a declared strength.

Reaction conditions

| | |
|---|---|
| Temperature | 37° C. |
| pH | 7.15 |
| Wavelength | 405 nm |
| Reaction time | 5 min |
| Measuring time | 2 min |

A folder (EB-SM-0216.02) describing this standard method in more detail is available on request from Novozymes A/S, Denmark, which folder is hereby included by reference.

### Xylose/Glucose Isomerase Assay (IGIU)

1 IGIU is the amount of enzyme which converts glucose to fructose at an initial rate of 1 micromole per minute at standard analytical conditions.

Standard Conditions:

Glucose concentration: 45% w/w

pH: 7.5

Temperature: 60° C.

Mg2+ concentration: 99 mg/l (1.0 g/l MgSO4*7H₂O)

Ca2+ concentration <2 ppm

Activator, SO₂ concentration: 100 ppm (0.18 g/l Na₂S₂O₅)

Buffer, Na₂CO₃, concentration: 2 mM Na₂CO₃

### Protease Assay Method—AU(RH)

The proteolytic activity may be determined with denatured hemoglobin as substrate. In the Anson-Hemoglobin method for the determination of proteolytic activity denatured hemoglobin is digested, and the undigested hemoglobin is precipitated with trichloroacetic acid (TCA). The amount of TCA soluble product is determined with phenol reagent, which gives a blue color with tyrosine and tryptophan.

One Anson Unit (AU-RH) is defined as the amount of enzyme which under standard conditions (i.e., 25° C., pH 5.5 and 10 min. reaction time) digests hemoglobin at an initial rate such that there is liberated per minute an amount of TCA soluble product which gives the same color with phenol reagent as one milliequivalent of tyrosine.

The AU(RH) method is described in EAL-SM-0350 and is available from Novozymes A/S Denmark on request.

### Protease Assay Method (LAPU)

1 Leucine Amino Peptidase Unit (LAPU) is the amount of enzyme which decomposes 1 microM substrate per minute at the following conditions: 26 mM of L-leucine-p-nitroanilide as substrate, 0.1 M Tris buffer (pH 8.0), 37° C., 10 minutes reaction time.

LAPU is described in EB-SM-0298.02/01 available from Novozymes A/S Denmark on request.

### Determination of Maltogenic Amylase Activity (MANU)

One MANU (Maltogenic Amylase Novo Unit) may be defined as the amount of enzyme required to release one micro mole of maltose per minute at a concentration of 10 mg of maltotriose (Sigma M 8378) substrate per ml of 0.1 M citrate buffer, pH 5.0 at 37° C. for 30 minutes.

### EXAMPLES

### Example 1

This example shows the laboratory simulation of the method of the invention. The results are compared with those from the control, which used lab vortex mixer to mix high-solids slurry.

### Simulation of the Mixing Method in Laboratory

Eight 50 mL VWR centrifuge tubes were aligned as hydrolysis reactors (Label R1-R8). To each tube 7.14 g of washed dilute acid-catalyzed steam explosion corn stover (dry matter (DM) 29.5 wt. %), 5.98 g of DI water, 2 mL of 1 M citrate buffer and 0.1 mL of 1 g/L penicillin was added. Cellulolytic Preparation A was diluted ten times to make 0.1× enzyme solution. The 0.1× enzyme solution contained 25.7 FPU/mL.

The simulation process started with the addition of 24 g DI water and 0.78 mL of 0.1× enzyme solution to reactor R3 to reach 5 wt % solids (low solids) and an enzyme dose of 10 FPU/g-DM. The low-solids reactor was then vortexed for 1 min followed by centrifugation at 3,700 rpm, 20° C., for 5 min. Twenty-four mL of the supernatant was transferred to R4 after centrifugation, by which the solids content in R3 was increased to 12.5 wt. % (high solids). R3 was then vortexed for 20 seconds and incubated in a water bath (50° C., 150 rpm).

To reactor R4, 0.78 mL 0.1× enzyme solution was added, followed by vortexing for 1 min and centrifugation at 3,700 rpm, 20° C., for 5 min. Twenty-four mL of the supernatant was transferred to R5. R4 was vortexed for 20 seconds and put it in 50° C., 150 rpm water bath.

The operations were repeated for R6-R8 and ended with collection of the supernatant of R8 in a beaker. The collected supernatant from R8 was analyzed with HPLC. The solid contents in all reactors (R3-R8) were 12.5 wt. % after the above operations;

Control assays were prepared by adding 0.78 mL enzyme solution to R1 and R2, vortexing for 1 min. The control reactors had the same solids content as R3-R8 and a predetermined enzyme dose of 10 FPU/g-dry solids. The average values from R1 and R2 were used.

### Sampling and Analysis

At 24 hrs and 72 hrs, three mL of slurry was taken and filtered through 0.2 micro meter membrane, acidify with 1% of 40% H₂SO₄ and dilute five times with 5 mM H₂SO₄ for HPLC analysis. An Agilent HPLC system equipped with Aminex HPX-87H column (Bio-Rad Laboratories, Hercules, Calif.) running at 0.6 mL/min of 5 mM H₂SO₄ (pH ˜1.8) was used for sugar quantification by integration of signal from a refractive index detector, based on calibration by pure sugar standard mixtures. Carbohydrate analysis of the washed PCS substrates was performed following the NREL Standard Analytical Protocols: Determination of Structural Carbohydrates and Lignin in Biomass (June 2007; Website: http://www.nrel.gov/biomass/analytical_procedures.html). This procedure uses a two-step acid hydrolysis to fractionate the carbohydrate polymers into monomeric sugars that are easily quantified on HPLC. Moisture content was determined using an IR-200 moisture analyzer (Denver Instrument Company, Denver, Colo.). The samples were heated at 130° C. in the moisture analyzer until less than 0.05% of initial weight was lost within 1 min. The total weight loss was taken as the moisture content in the samples.

### Results

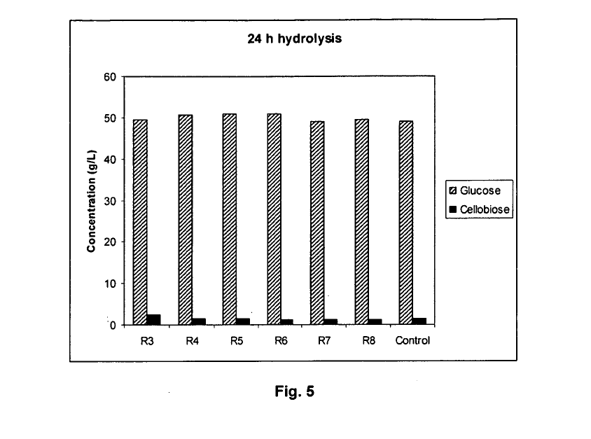

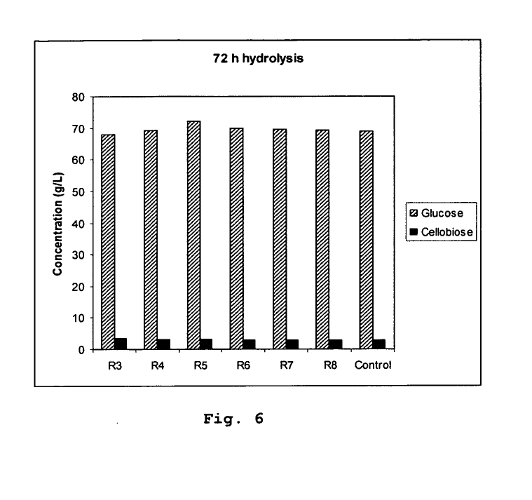

FIGS. 5 and 6 show the glucose and cellobiose concentrations in the simulation reactors (R3-R8) as well as in the controls (R1 and R2, average values used). The relative deviations (CV) of the glucose concentrations obtained from the controls were below 1%. Both the glucose and cellobiose concentrations in R8 were equal to those from the controls either at 24 hrs or at 72 hrs, which indicates that the enzyme was efficiently distributed in the slurry. Moreover, no significant differences were seen in the glucose concentrations of R3-R8, and in the cellobiose concentrations of R4-R8. This indicates that when using Cellulolytic preparation A for lignocellulose hydrolysis according to the method of the invention, a steady state can be reached shortly after the startup of the operation.

HPLC analysis of the supernatant from R8 showed the sugar concentrations (g/L) in the liquid as: cellobiose, 0.075; glucose, 0.575; xylose, 0.115; arabinose, 0.02. These data indicates that negligible hydrolysis took place in the low-solids stage.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties. The present invention is further described by the following examples which should not be construed as limiting the scope of the invention.

1-20. (canceled)

21. A method for treating pre-treated lignocellulose-containing material comprising the steps of:
a) subjecting a high solids slurry comprising pre-treated lignocellulose-containing material to dilution and agitation in the presence of one or more chemicals and/or one or more enzymes;
b) subjecting the treated slurry from step a) to liquid-solid separation;
c) recycling at least a portion of the liquid separated from step b) for diluting a slurry as described in step (a);
d) optionally transferring the solids-containing material coming from step b) for downstream processing.

22. The method of claim 21, further comprising:
e) subjecting all or part of the solids-containing material obtained from the liquid-solid separation in step b) or f) to dilution and agitation in the presence of one or more chemicals and/or one or more enzymes;
f) subjecting the material from step e) to liquid-solid separation;
g) recycling at least a portion of the liquid separated from step f) for diluting a slurry as described in step (a) or the solids-containing material in step e); and
h) transferring the solids-containing material coming from step f) for downstream processing.

23. The method of claim 21, wherein agitation is carried out in a mixing tank, vessel, pump or the like.

24. The method of claim 21, wherein the enzymes added during step a) or e) are hydrolytic enzymes.

25. The method of claim 21, wherein the lignocellulose-containing material in step a) or e) constitutes from between 0.5-15 wt. % of the slurry determined as insoluble solids prior to dilution.

26. The method of claim 21, wherein at least a portion of the liquid separated in step b) or f) is recycled to step a) or step e).

27. The method of claim 21, wherein the hydrolytic enzymes are selected from the group consisting of amylolytic enzymes cellulolytic enzymes, esterase enzymes, hemicellulolytic enzymes, pectolytic enzymes, proteolytic enzymes, or a mixture of two or more thereof.

28. The method of claim 21, wherein the concentration of lignocellulose-containing material in the slurry in step a) or step e) is adjusted with the recycled liquid from step b) or step f).

29. The method of claim 21, wherein liquid-solid separation in step b) or f) is done using a centrifuge, belt press, drum filter, hydrocyclone or filter press.

30. The method of claim 21, wherein from 40-99 wt. % liquid of the total solids and liquids separated in step b) or f) is recycled.

31. The method of claim 21, wherein the lignocellulose-containing material leaving step b) or f) constitutes from between 10-80 wt. % of the total solids and liquid as determined by insoluble solids.

32. The method of claim 21, wherein a hemicellulolytic enzyme is added during treatment in step a) or during step e).

33. The method of claim 21, wherein the solids-containing material from step b) or step f) is transferred for downstream processing, wherein the downstream processing comprises simultaneous hydrolysis and fermentation, hybrid hydrolysis and fermentation, or separate hydrolysis and fermentation.

34. The method of claim 21, wherein the recycled liquid coming from solid-liquid separation in step b) or step f) is subjected to conditioning.

35. A process of producing a fermentation product from lignocellulose-containing material comprising the steps of:
i) pre-treating lignocellulose-containing material;
ii) subjecting the pre-treated material using a method of treating pre-treated lignocellulose-containing material as defined in claim 21;
iii) hydrolyzing the material coming from step ii); and
iv) fermenting using one or more fermenting organisms.

36. The process of claim 35, wherein the treatment in step ii) and fermentation in step iii) is carried out as simultaneous hydrolysis and fermentation, hybrid hydrolysis and fermentation, or separate hydrolysis and fermentation.

37. The process of claim 35, wherein the lignocellulose-containing material is chemically, mechanical or biologically pre-treated in step i).

38. The process of claim 35, wherein the fermentation product is ethanol.

39. The process of claim 35, wherein the fermenting organism is yeast.

40. The process of claim 35, wherein the lignocellulose-containing material is derived from corn cobs, corn fiber, corn stover, rice straw, wheat straw, pine wood, wood chips, hardwood, softwood, switchgrass, Miscanthus, rice hulls, municipal solid waste, industrial organic waste, office paper, bagasse, paper and pulp processing waste, or mixtures thereof.
